(19) United States
(12) Patent Application Publication
(10) Pub. No.: US 2017/0014579 A1
(43) Pub. Date: Jan. 19, 2017

(54) BLUNT CANNULA NEEDLE DEVICE

(71) Applicant: SMITHS MEDICAL ADS, INC., Plymouth, MN (US)

(72) Inventor: Troy M. Derby, Stoddard, NH (US)

(73) Assignee: SMITHS MEDICAL ASD, INC., Plymouth, MN (US)

(21) Appl. No.: 15/189,181

(22) Filed: Jun. 22, 2016

Related U.S. Application Data
(60) Provisional application No. 62/192,362, filed on Jul. 14, 2015.

(51) Int. Cl. A61M 5/32; A61J 1/20; A61M 5/28; A61M 5/34

(52) U.S. Cl. CPC A61M 5/329; A61M 5/3202; A61M 5/343; A61J 1/201; A61M 5/288

(57) ABSTRACT

A needle device has a needle assembly including a hub having one end fitted with a blunt cannula. The blunt cannula has a base section, a central section and a shaft having a distal apertured end. The needle of the needle assembly extends through an internal passage of the blunt cannula and is sealingly fitted to a portion of the internal passage. The hub of the needle assembly has another end connectable to a syringe. When the device is connected to a syringe, a fluid communication path is established between the apertured end of the shaft and the interior of the syringe via the lumen of the needle. The shaft of the blunt cannula is usable to pierce the seal of a fluid store while maintaining the needle in an unused state for subsequent patient use. The distal end of the shaft may be protected by an end cap.

### FIELD OF INVENTION

The instant invention relates generally to medical devices, and more particularly to a needle device adaptable for use with a syringe that combines a blunt cannula with a needle to enable access to a medicament while maintaining the needle in an unused condition for subsequent first use on a patient.

### BACKGROUND OF THE INVENTION

In a hospital or medical environment, well known medical devices or components are typically used to deliver a medical solution or medicament to a patient. In one typical patient medical solution delivery procedure, a syringe, a hypodermic needle and a blunt cannula are cooperatively used in a discrete and separate sequence of steps to draw a desired medicament from a vial or container into a syringe for subsequent delivery to the patient.

Typically, a blunt cannula is attached to a syringe via a luer lock or luer slip. The blunt cannula is used to pierce a rubber stopper or septum that seals a vial. When the tip of the blunt cannula is in fluid access with the medicament stored inside the vial, a desired amount or volume of the medicament may be drawn into the syringe barrel. The blunt cannula is then removed and a new unused hypodermic needle is attached to the syringe to inject the medicament into a patient. In this known procedure, the blunt cannula and needle each are removable from and attachable to the syringe. A drawback of this procedure is that the attachment and interchange of the blunt cannula and needle to the syringe involves multiple steps that require more time to complete. Additionally, there is an increased cost since both a blunt cannula and a hypodermic needle are needed for this procedure.

In another medicament delivery procedure, a single new and unused hypodermic needle is attached to a syringe. The needle is first used to pierce the rubber stopper that seals the vial and to withdraw the medicament stored in the vial. The same needle is thereafter used to prick the patient to inject the withdrawn medicament into the patient. By using the same needle, this procedure eliminates the above-described needle interchange step and also the need to purchase and use a blunt cannula or multiple needles. However, since the needle has to first pierce through a rubber stopper, the fragile hypodermic needle tip may be deformed and/or damaged, the sharpness of the needle tip dulled, and the surface silicone coating that protects the needle possibly removed. The pricking with such blemished needle is more often than not painful to the patient.

Therefore, there exists a need for a needle device usable to access a medicament from a sealed fluid store but yet maintains the needle of the device in a new or unused condition for delivery of the medicament to a patient.

### BRIEF SUMMARY OF THE INVENTION

The instant invention has a blunt cannula and a needle assembly configured to couple to each other to form a medical needle device adapted to access and withdraw a medicament from a sealed fluid store such a vial and to present a non-blemished needle for pricking and injecting the withdrawn medicament to a patient.

The blunt cannula has a base section having a receptacle end configured to be connectable to a complementary configured distal portion of the needle hub of a needle assembly and a shaft with an apertured end that encloses the sharp tip of the needle that extends from the needle hub of the needle assembly. A central section of the blunt cannula has a portion configured to have a passage dimensioned to sealaingly fit with the needle so that a seal is formed between at least respective portions of the outer wall of the needle and the inner wall of the blunt cannula. Accordingly, once the blunt cannula is fully coupled to the needle assembly, a fluid communication path is established between the aperture at the distal end of the shaft and through the lumen defined by the needle to the connector end of the needle hub of the needle assembly that is adapted to connect to a syringe.

With the tip of the needle covered by the distal apertured end of the blunt cannula, the integrity of the needle tip remains intact as the distal apertured end of the shaft of the blunt cannula is used to pierce the septum or rubber stop that seals the vial. Once the distal apertured end of the shaft reaches the medicament stored in the vial, with the needle assembly connected to a syringe, the desired amount of the medicament may be drawn into the syringe barrel. Thereafter, the shaft is removed from the vial, recapped with an end cap that previously caps the distal apertured end of the shaft prior to use. To use the inventive device on a patient, the capped blunt cannula is removed from the needle assembly to expose the unblemished needle which may then be used to prick the patient and to deliver the medicament stored in the syringe to the patient.

The present invention is therefore directed to a medical device comprising: a blunt cannula having a base section adapted to couple to a needle hub, a central section distally adjacent to the base section having a longitudinal internal central passage, a shaft extending distally from the central section, the shaft having an internal shaft passage in alignment with the central passage along a longitudinal axis; and a needle assembly having a needle extending from a needle hub; wherein the shaft passage and the central passage are configured to sealingly receive the needle from the needle assembly so that a seal fit is formed between the needle and the shaft and central passages when the needle assembly and the blunt cannula are coupled to each other; and wherein the needle assembly with the blunt cannula coupled thereto is adapted to be attached to a syringe for use with a fluid store.

The present invention is further directed to a medical device comprising: a blunt cannula having a proximal end and a distal end, a base section having an opening at the proximal end, a shaft having a distal apertured end that forms the distal end of the blunt cannula, and a central section sandwiched between the base section and the shaft, an internal passage connecting the proximal and distal ends of the blunt cannula; and a needle assembly having a needle hub including a distal end where a needle defining a lumen having a sharp end extends and an open proximal end coupleable to a syringe; wherein when the blunt cannula and the needle assembly are assembled together with the distal end of the needle hub fittingly mated to the opening at the base of the blunt cannula, the needle is slidingly positioned in the passage with the sharp end of the needle positioned proximally from the distal apertured end of the shaft and at least a portion of the passage and the needle forming a seal along the passage of the blunt cannula to establish a fluid path between the apertured end of the shaft and the proximal end of the needle hub; and wherein when a syringe is coupled to the proximal end of the needle assembly to be in communication with the fluid path and the apertured end of the shaft is in a fluid store having a fluid, the fluid may be drawn into the syringe via the fluid path.

The present invention is also directed to a method for providing to a patient a medicament from a sealed fluid store comprising the steps of:
(a) coupling a blunt cannula having a shaft including a distal apertured end to a needle assembly having a needle with a sharp end to form a blunt cannula needle device where the needle is sealingly fitted to at least a portion of the shaft with the needle defining a lumen;
(b) attaching the blunt cannula needle device to a syringe having a plunger;
(c) piercing the sealed fluid store with the distal apertured end of the shaft;
(d) inserting the shaft sufficiently into the fluid store so that the distal apertured end is in fluid communication with a medicament stored in the fluid store;
(e) retracting the plunger to draw a desired amount of the medicament from the fluid store into the syringe via the shaft and the needle lumen;
(f) removing the shaft of the blunt cannula from the fluid store;
(g) separating the blunt cannula from the needle assembly to expose the needle;
(h) pricking the patient with the needle to positioned the tip of the needle in a desired location in the patient; and
(I) moving the plunger to inject the medicament through the needle to the patient.

The present invention is moreover directed to a medical device, comprising: a needle assembly including a hub having an open end connectable to a syringe and a closed end wherefrom a needle defining a lumen extends, and a blunt cannula having an distal apertured end and a receptacle end fittingly coupled to the closed end of the hub; wherein the distal apertured end of the blunt cannula is usable to pierce a septum of a fluid store to establish a fluid path between the fluid store and the syringe via the distal apertured end of the blunt cannula and the needle lumen when the needle assembly is connected to the syringe.

### BRIEF DESCRIPTION OF THE DRAWINGS

The invention will become apparent and may be best understood with reference to the following detailed description of the invention in connection with the accompanying drawings, in which:

FIG. 1 is a perspective view of a blunt cannula needle device;

FIG. 2 is a partially exploded perspective view of the blunt cannula needle device of FIG. 1;

FIG. 3 is an exploded perspective view of the blunt cannula needle device shown in FIG. 1;

FIG. 4 is a side view of the blunt cannula needle device of FIG. 1;

FIG. 5 is a cross sectional side view along section line A-A of the blunt cannula needle device shown in FIG. 1;

FIG. 6 is an illustration of the blunt cannula needle device of FIG. 1 with its end cap removed and having been attached to a syringe in the process of interfacing with a sealed vial;

FIG. 7 is an illustration showing the blunt cannula needle device of FIG. 6 prior to recapping of the blunt cannula; and

FIG. 8 is an illustration showing the blunt cannula needle device of FIG. 7 with the blunt cannula removed from the needle assembly to expose the hypodermic needle.

### DETAILED DESCRIPTION OF THE INVENTION

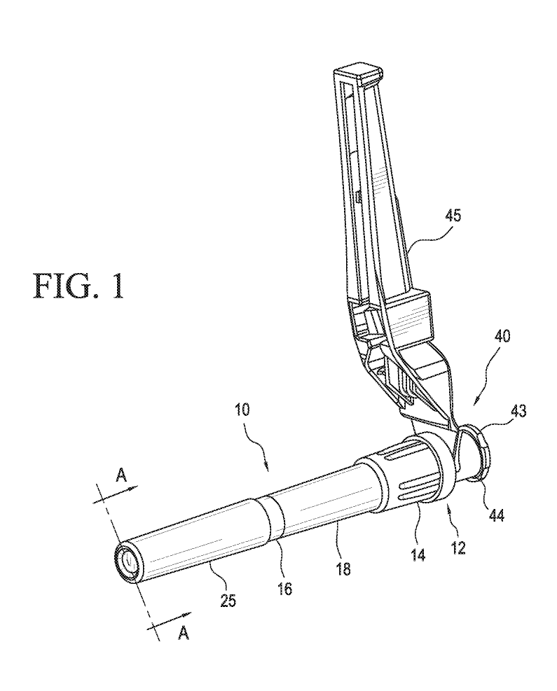

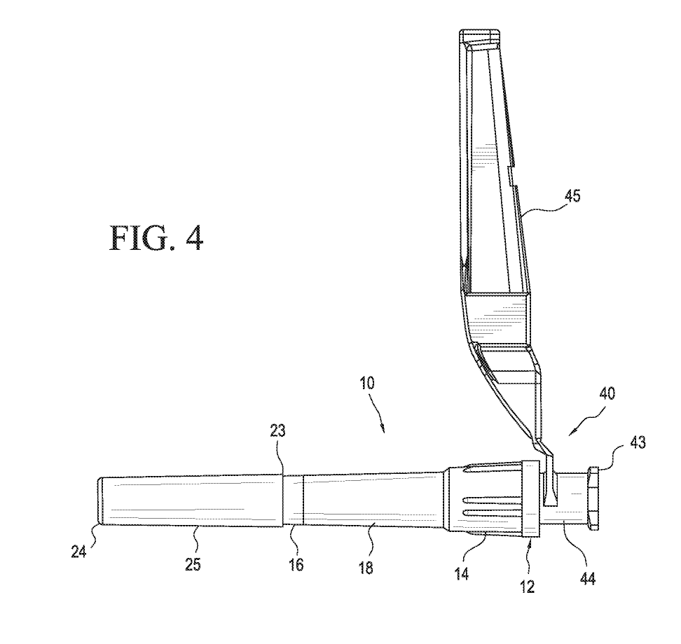

With reference to FIGS. 1-5, the inventive blunt cannula needle device is shown to have a blunt cannula 10 having a proximal base section 14, a central section 18 and a distal cannula shaft 26 longitudinally and concentrically disposed between a proximal end 12 and a distal end 17. Blunt cannula 10 is operatively attached or coupled to a needle assembly 40 which includes a needle hub 44, a needle 48 having a sharp bevel needle tip 49 that angles to a needle heel 46 and a protective needle sheath 45 that may be used to fixedly hold and cover a used or contaminated needle by an internal hook as described in U.S. Pat. No. 8,603,042. The disclosure of the '042 patent is incorporated by reference herein.

A lumen formed by the shaft of the needle extends from needle tip 49 at the distal end 47 of needle assembly 44 through needle hub 44 to a chamber 41 that opens to the proximal end of the needle assembly, referenced by a circumferential or semi-circumferential flange 43 of a connector as is conventionally known. The needle assembly connector of the exemplar embodiment may be a conventional luer or a non-luer connector.

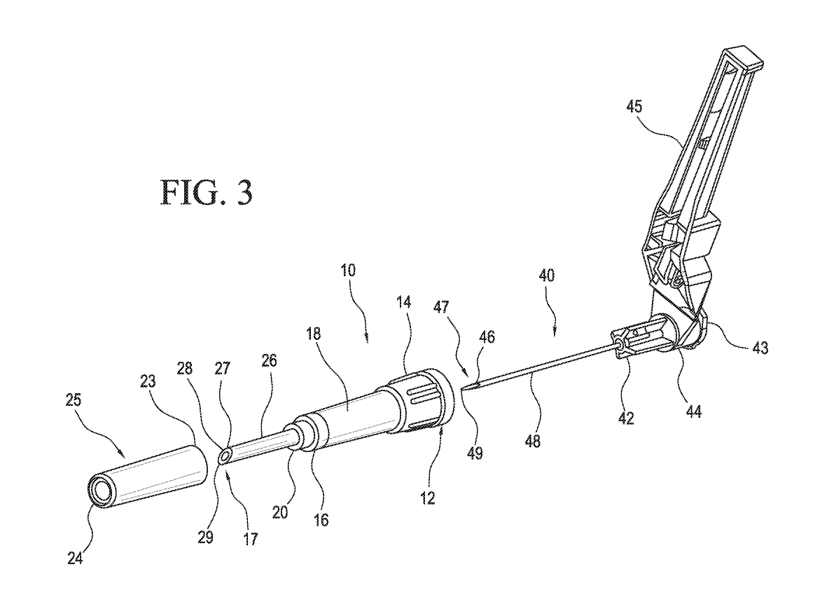

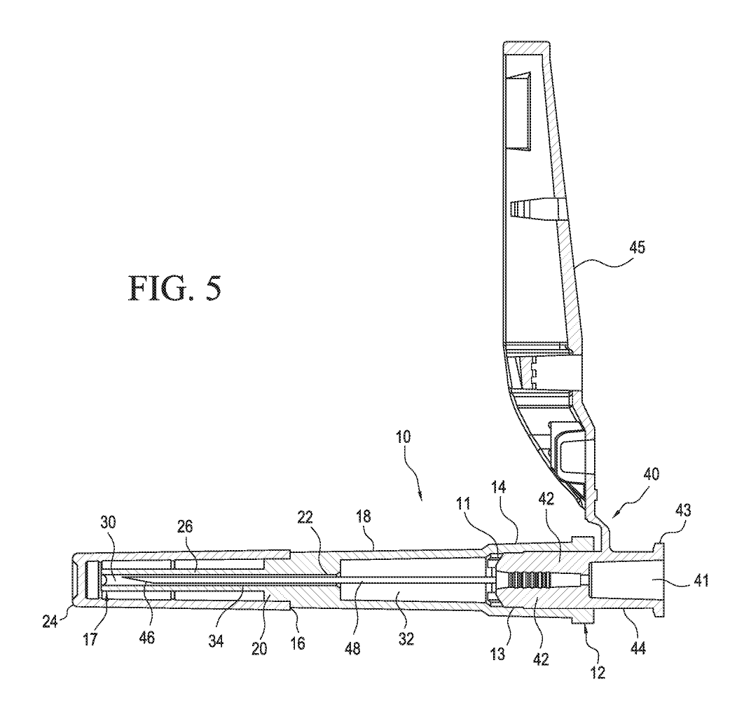

Base section 14 of blunt cannula 10 extends distally from proximal end 12 and has a cavity 13 adapted to fittingly mate with a distal portion of needle hub 44 of needle assembly 40. As best show in FIG. 5, the base of cavity 13 of base section 14 abuts against a circumferential shoulder 11, which acts as a stop, at the distal end of needle hub 44 when blunt cannula 10 is fully mated to the distal portion of needle hub 44. As shown in FIG. 3, the distal portion of needle hub 44 has a plurality of spaced apart longitudinal ribs, tabs or extensions 42 that extend orthogonal from needle 48. The circumferential wall of cavity 13 and the respective longitudinal edges of the extensions 42 are configured to provide a friction fit between base section 14 and the distal portion of needle hub 44.

Central section 18 of blunt cannula 10 has an internal central passage 22 extending longitudinally through its distal portion and a through bore 32 at its center and proximal portions, as per shown in FIG. 5. Passage 22 is dimensioned to accept needle 48 of the needle assembly 40. The respective cross dimensions of needle 48 and passage 22 are configured to enable the outer circumferential surface of needle 48 and the inner circumferential wall of passage 22 to movably and yet sealingly engage or fit to each other so as form a seal fit 34 therebetween when blunt cannula 10 is coupled to needle assembly 40 and at least a portion of needle 40 is positioned along passage 22. In the exemplar embodiment shown in FIG. 5, seal fit 34 extends along a portion of the length of passage 22 as well as a portion of shaft 26 of blunt cannula 10.

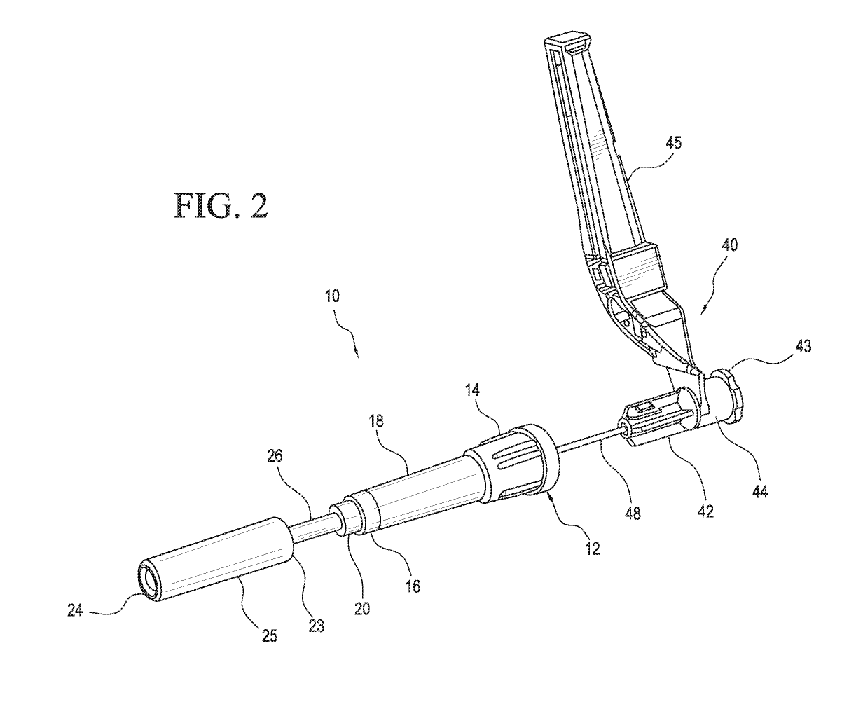

A stepped external circumferential shoulder 16 and a projection 20 are formed at the junction where central section 18 and shaft 26 meet. As shown in FIGS. 2-3 and 5-7, shaft 26 is covered by an end cap 25 that has a closed end and an open end 23. Projection 20 and open end 23 are respectively dimensioned so that end cap 25 may be frictionally held to projection 20. When fully fitted to projection 20, the open end of cap 25 abuts shoulder 16 to prevent further coupling movement between end cap 25 and blunt cannula 10.

Shaft 26 is concentric to and extends distally from central section 18 to distal end 17 of blunt cannula 10. Shaft 26 has an internal longitudinal passage 30 and a distal apertured end 28 that may include a bevel tip 19 that slopes to a heel 27. Shaft passage 30 is a continuous extension of passage 22 of central section 18. Thus, blunt cannula 10 has a through passage that connects the distal apertured end 28 and bore 32 at the proximal portion of central section 18, as per shown in FIG. 5. For sake of simplicity, the respective passages 30 and 22 of shaft 10 and central section 18 may be referred to as the longitudinal or through passage of the blunt cannula. Also, it should be appreciated that the aperture at the distal end of the shaft does not necessarily have to be an opening at the distal end of the shaft, as the opening may in practice be at a side, or both sides, at the distal end of the shaft so that the tip of the shaft may be a sharp end that facilitates the piercing of an elastomer septum or rubber stop that covers a fluid store, as will be discussed in greater detail, infra.

In the exemplar embodiment shown in FIGS. 1-5, base section 14, central section 18 and shaft 26 are formed or molded together from a medical plastics material so that blunt cannula 10 is a single integral unitary piece or component.

When needle assembly 40 is fully coupled with blunt cannula 10 such that needle 48 is fittingly threaded along passages 22 and 30, or the through passage of the blunt cannula, the apertured end of shaft 26 is positioned distally from the sharp bevel tip 29 of needle 48. As the outer wall of needle 48 and the inner wall of the through passage are sealingly engaged to each other to prevent fluid from passing therebetween, a fluid communication path that enables the passing of a fluid between apertured end 28 of shaft 26 and chamber 41 at the connector end of needle assembly 40 is established from passage 30 at the distal portion of shaft 26 through the lumen defined by needle 48 that extends to chamber 41 of needle hub 44.

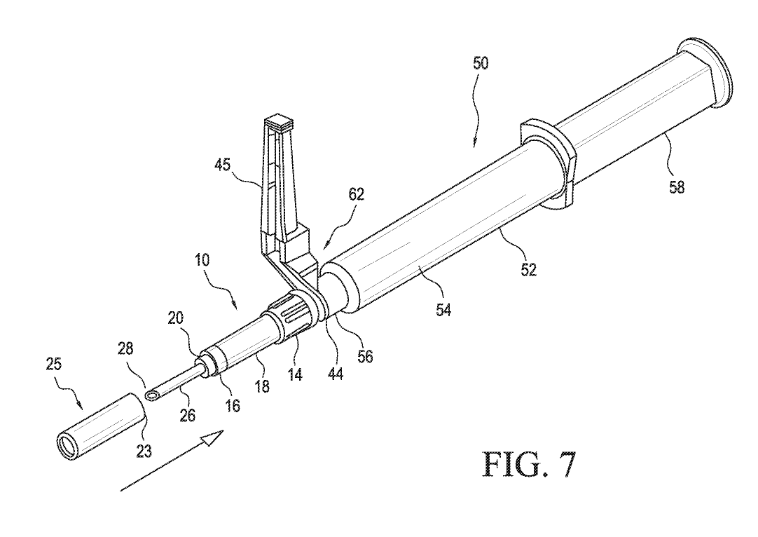

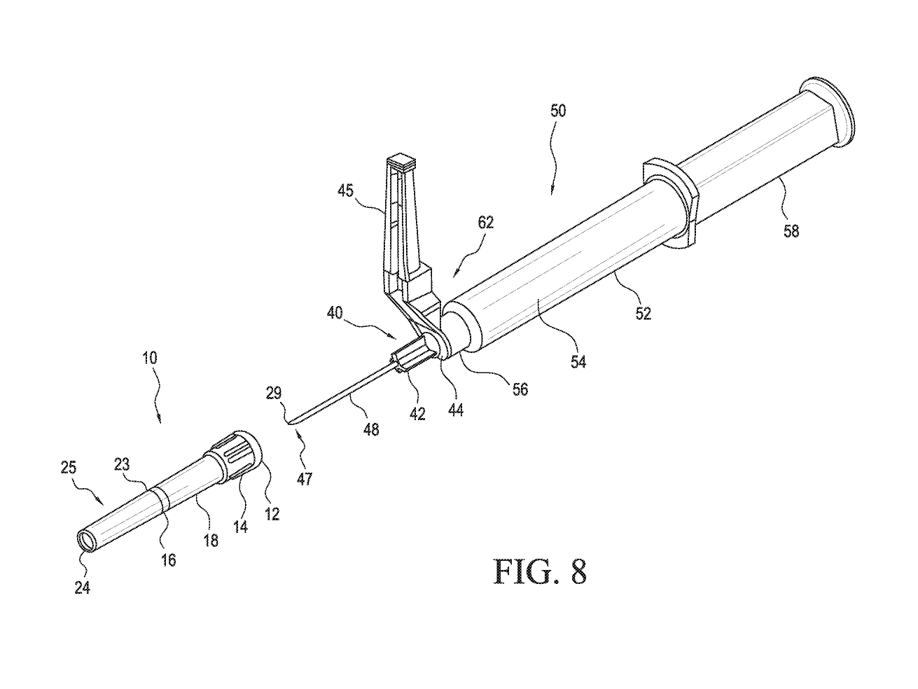

As shown in FIGS. 1, 4 and 5-8, the blunt cannula 10 is operatively coupled or attached to needle assembly 40, which in turn is coupled to a syringe 50 having a barrel 52, a chamber 54 for storing fluid, a connector 56 at its distal end 62 and a plunger 58 inserted to and movable along barrel 52 as is conventionally known. Also as is well known, when fully assembled as per shown in FIG. 7, a fluid may be drawn into or expelled from the chamber of the syringe by reciprocal movements of plunger 58 relative to barrel 50. Connector 56 of the syringe may be a conventional luer, although a non-luer connector including the CORRECTINJECT (CI) connector of the assignee is also envisioned.

FIGS. 1-3 and 6-8 illustrate a preferred method of using the inventive device for accessing a medicament stored in a fluid store such as a vial 70 sealingly capped with a rubber stopper or elastomeric septum. As to be described below, the blunt cannula needle device of the instant invention enables access to the medicament 74 in vial 70 while maintaining the hypodermic needle 40 in a new or unused condition for subsequent injection of the retrieved medicament to a patient.

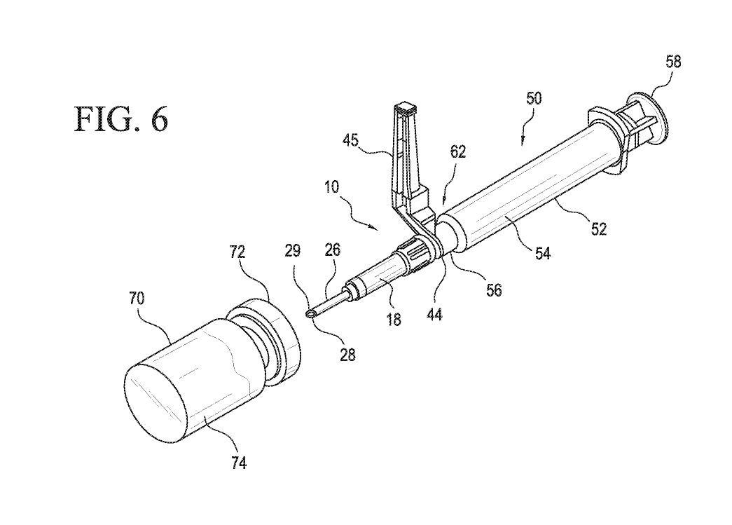

As shown in FIG. 6, in preparation for accessing the medicament 74 stored in vial 70, end cap 25 is removed from blunt cannula 10. The distal apertured end 28 of shaft 26 is then used to pierce the elastomeric septum 72 that seals vial 70. Since needle 48 is housed in shaft 26 and is positioned behind the apertured end 28 and therefore does not come into contact with the rubber seal when the seal is being punctured, there is no adverse effect to the sharp tip of needle 48. After the septum is punctured, shaft 26 is moved into vial 70 until distal apertured end 28 comes into fluid communication with the medicament stored in the vial. As the desired amount of medicament 74 is withdrawn from vial 70, seal fit 34 established between shaft 26 and needle 48 prevents any seepage of the medicament at blunt cannula 10 so that the medicament is guided by the passage of shaft 26 between its distal apertured end and the distal opening of the lumen defined by needle 48, through the lumen to chamber 41 of needle assembly 40 and from there conveyed to syringe chamber 54.

As shown in FIG. 7, after the desired amount of medicament is withdrawn into syringe chamber 54, shaft 26 of blunt cannula 10 is removed from vial 70 and shaft 26 is recapped with end cap 25. To use on a patient, blunt cannula 10 with its shaft 26 covered by end cap 25 is removed from needle assembly 40 to expose needle 48. The uncovered needle 48 may then be used to prick the patient so that the tip of the needle may be appropriately located in the patient and the medicament stored in the syringe may then be delivered to the desired site, such as a vein, an artery or muscle, of the patient. After use, sheath 45 is pivoted into longitudinal alignment with the needle assembly 40 to cover the now contaminated needle 48. At least one internal hook in sheath 45 grasps needle 48 so that the sheath non-removably covers the needle. Thus, with the inventive blunt cannula needle device, the needle used to prick the patient remains in a new, unblemished state throughout the process of withdrawing the medicament from a fluid store.

The embodiments of the invention described above are intended to be illustrative only and not limiting. It should therefore be recognized that changes may be made in form and detail without departing from the spirit and scope of the subject matter.

1. A medical device comprising:
a blunt cannula having
a base section adapted to couple to a needle hub;
a central section distally adjacent to the base section having an internal central passage;
a shaft extending distally from the central section, the shaft having an internal shaft passage in alignment with the central passage along a longitudinal axis to form a longitudinal passage along the blunt cannula; and
a needle assembly having a needle extending from a needle hub;
wherein the longitudinal passage is configured to sealingly receive the needle from the needle assembly so that a seal fit is formed between the needle and the longitudinal passage at at least a portion thereof when the needle assembly and the blunt cannula are coupled to each other; and
wherein the needle assembly with the blunt cannula coupled thereto is adapted to be attached to a syringe for use with a fluid store.

2. The device of claim 1,
wherein the shaft of the blunt cannula comprises an apertured end; and
wherein the needle comprises a sharp distal end;
whereby the apertured end is positioned distally from the sharp distal end when the needle is appropriately positioned within the shaft passage.

3. The device of claim 1, further comprising an end cap having a distal closed end and a proximal open end; and
wherein the central section of the blunt cannula comprises at its distal portion an outer circumferential shoulder and a projection that extends distally from the shoulder;
whereby the end cap is adapted to fittingly couple to the projection via its open end to enclose the shaft of the blunt cannula.

4. The device of claim 1, wherein the base section, the central section and the shaft of the blunt cannula are molded to form a one piece integral component.

5. The device of claim 1, wherein the base section, the central section and the shaft are parts of a single integral component with the longitudinal passage extending therethrough, wherein when the sharp distal end of the needle is positioned in the shaft of the blunt cannula, a fluid communication path is established through the lumen of the needle between the shaft and the needle hub of the needle assembly.

6. The device of claim 1, wherein the base section, the central section and the shaft of the blunt cannula are made from a plastics material.

7. The device of claim 1, wherein the fluid store is a vial having stored therein a fluid medicament.

8. A medical device comprising:
a blunt cannula having a proximal end and a distal end, a base section having an opening at the proximal end, a shaft having a distal apertured end at the distal end of the blunt cannula, and a central section sandwiched between the base section and the shaft, an internal passage connecting the proximal and distal ends of the blunt cannula;
a needle assembly having a needle hub including a distal end where a needle defining a lumen having a sharp end extends and an open proximal end coupleable to a syringe;
wherein when the blunt cannula and the needle assembly are assembled together with the distal end of the needle hub fittingly mated to the opening at the base of the blunt cannula, the needle is slidingly positioned in the passage with the sharp end of the needle positioned proximally from the apertured end of the shaft and at least a portion of the passage and the needle forming a seal along the passage of the blunt cannula to establish a fluid path through the lumen between the apertured end of the shaft and the proximal end of the needle hub; and
wherein when a syringe is coupled to the proximal end of the needle assembly to be in communication with the fluid path and the apertured end of the shaft is in a fluid store in fluid communication with a fluid stored therein, the fluid may be drawn into the syringe via the fluid path.

9. The device of claim 8, further comprising an end cap having a distal closed end and a proximal open end; and
wherein the central section comprises an outer circumferential shoulder and a projection that extends distally from the shoulder;
whereby the end cap is adapted to fittingly couple to the projection via its open end to enclose the shaft.

10. The device of claim 8, wherein the base section, the central section and the shaft are molded to form a single integral component.

11. The device of claim 8, wherein the base section, the central section and the shaft are parts of a single integral component; and
wherein when the sharp distal end of the needle is positioned in the shaft of the blunt cannula, a fluid communication path is established through the lumen of the needle between the apertured end of the shaft and the needle hub of the needle assembly.

12. The device of claim 8, wherein the blunt cannula is made of a plastic material.

13. The device of claim 8, wherein the fluid store is a drug vial and the medicament is a fluid.

14. A method for providing to a patient a medicament from a sealed fluid store comprising:
(a) coupling a blunt cannula having a shaft including a distal apertured end to a needle assembly having a needle with a sharp end to form a blunt cannula needle device where the needle is sealingly fitted to at least a portion of the shaft with the needle defining a lumen;
(b) attaching the blunt cannula needle device to a syringe having a plunger;
(c) piercing the sealed fluid store with the distal apertured end of the shaft;
(d) inserting the shaft sufficiently into the fluid store so that the distal apertured end is in fluid communication with a medicament stored in the fluid store;
(e) retracting the plunger to draw a desired amount of the medicament from the fluid store into the syringe via the shaft and the needle lumen;
(f) removing the shaft of the blunt cannula from the fluid store;
(g) separating the blunt cannula from the needle assembly to expose the needle;
(h) pricking the patient with the needle to positioned the tip of the needle in a desired location in the patient; and
(I) moving the plunger to inject the medicament through the needle to the patient.

15. The method of claim 14, further comprising
capping the blunt cannula with an end cap after step (f); and
detaching the capped blunt cannula from the needle assembly prior to step (h).

16. A medical device, comprising:
a needle assembly including a hub having an open end connectable to a syringe and a closed end wherefrom a needle defining a lumen extends;
a blunt cannula having a distal apertured end and a receptacle end fittingly coupled to the closed end of the hub;
wherein the distal apertured end of the blunt cannula is usable to pierce a septum of a fluid store to establish a fluid path between the fluid store and the syringe via the apertured end of the blunt cannula and the needle lumen when the needle assembly is connected to the syringe.

17. The device of claim 16, further comprising a cap to cover the distal end of the blunt cannula.

18. The device of claim 16, wherein the blunt cannula comprises a body having a shaft having the distal apertured end, a through passage extending along the shaft and the body of the blunt cannula;
wherein the needle extending from the hub has a sharp distal end positioned proximally from the distal apertured end of the shaft of the blunt cannula when the blunt cannula is coupled to the hub.

19. The device of claim 16, wherein the blunt cannula protects the sharp end of the needle from contacting the septum when the septum is being pierced by the distal apertured end; and wherein the needle is usable to pierce a patient only after the blunt cannula is removed from the needle assembly.
